(19) United States
(12) Plant Patent Application Publication
(10) Pub. No.: US 2019/0307040 P1
(43) Pub. Date: Oct. 3, 2019

(54) Artichoke variety named 'PAGA 13-1'

(50) ;

(71) Applicant: Michael R. Scattini, Salinas, CA (US)

(72) Inventor: Michael R. Scattini, Salinas, CA (US)

(21) Appl. No.: 16/501,354

(22) Filed: Mar. 29, 2019

Related U.S. Application Data
(60) Provisional application No. 62/650,952, filed on Mar. 30, 2018.

(51) Int. Cl. A01H 6/14

(52) U.S. Cl. PLT/258

(57) ABSTRACT

A new and distinct variety of artichoke (Cynara cardunculus var. scolymus L.) plant named ‘PAGA 13-1’, particularly characterized by its high yield, large primary bud size, large number of secondary and tertiary buds, wide bracts with fleshy base, thick stems, and late maturity, is disclosed.

### CROSS REFERENCE TO RELATED APPLICATION

This application claims the benefit of U.S. Prov. App. No. 62/650,952, filed Mar. 30, 2018, which is hereby incorporated by reference in its entirety.

Latin name: Botanical classification: Cynara cardunculus L. var. scolymus.

Varietal denomination: Varietal denomination of the claimed artichoke variety: ‘PAGA 13-1’.

### BACKGROUND OF THE INVENTION

Artichoke (Cynara cardunculus L. var. scolymus), or globe artichoke, is a thistle-like perennial plant native to the Mediterranean region and is a member of the family Asteraceae. Artichoke belongs to the species Cynara cardunculus L. that includes two subspecies/cultivation groups: the cardoon (Cynara cardunculus L. var. altilis) that is cultivated for its edible leaf stems, and the artichoke that is cultivated for its edible immature inflorescence hereinafter referred to as buds, heads, or capitula. In some instances, artichoke is distinguished as Cynara scolymus.

Artichoke is a popular vegetable food source in many regions of the world, with nearly two million tons of artichokes produced annually. The United States produces approximately 40,000 tons of artichokes annually, with California being responsible for nearly 100% of its production, of which about 80% is grown in Monterey County.

Historically, a common perennial artichoke variety that is grown in the United States is ‘Green Globe’, also referred to as an “Heirloom” artichoke. Despite the use of it as a traditional cultivar, ‘Green Globe’ does not produce large numbers of large buds which are most desired by consumers. Further, ‘Green Globe’ exhibits a substantial amount of phenotypic variation as well as protracted and unpredictable harvest intervals.

Thus, there exists a need for improved artichoke varieties having large sized post-primary bud production, programmable and predictable harvest timing, and better uniformity. The present artichoke variety described herein is a product of the efforts to develop such artichoke varieties.

### BRIEF SUMMARY

The present disclosure relates to a new and distinct artichoke variety (Cynara cardunculus L. var. scolymus) which has been denominated as ‘PAGA 13-1’. This new artichoke variety exhibits increased bud yield, size and plant vigor compared to ‘Green Globe’, and produces a large primary bud, about 10-19 secondary buds, and about 10-16 tertiary buds per primary reproductive shoot. Up to 6 of the primary and secondary buds are 12-15 inches in circumference. Between 4-5 of the secondary and tertiary buds are 10¾-12½ inches in circumference or larger. The remaining buds are generally between 3-10½ inches in circumference. The plants produce between 4 and 10 reproductive shoots, in addition to the primary reproductive shoot, in the first production cycle. These secondary reproductive shoots produce less marketable sized buds than the primary shoot, although cultural practices such as cutting the primary reproductive shoot from the plant after harvest can materially increase the size and number of buds produced by these secondary shoots.

The new artichoke variety described herein is the product of an artichoke plant improvement program conducted over a number of years. The original parent is the unpatented Cynara cardunculus L. var. scolymus variety ‘Green Globe’, commonly known and cultivated in Castroville, Calif. for more than 90 years. This improvement project began with the objective to develop an artichoke variety with superior bud production. This included selecting for improvements in both commercial quality of buds, as well as overall bud consistency and large size bud production per plant. Additionally, plants were selected based on their predictable and condensed harvest phase.

The new artichoke variety described herein was discovered after several years of isolating and propagating artichoke variety ‘Green Globe’ in Castroville, Calif. From this continual propagation, several selections were identified which exhibited superior yield characteristics as compared to ‘Green Globe’. After identifying the superior quality plants from field conditions, the highest quality plants were selected for induction into laboratory tissue culture reproduction. These tissue-cultured plants were then planted in a test field in Castroville, Calif., where further selections were made among the population. Selections from this field were propagated by tissue culture, with subsequent field selections and tissue culture propagation conducted. The variety described herein as ‘PAGA 13-1’ is a final product obtained from these selections and tissue culture propagation cycles.

Artichoke variety ‘PAGA 13-1’ was subsequently asexually reproduced via tissue culture propagation and regeneration of artichoke plants from plantlets. The present variety has been found to be stable and reproduce true to type through successive asexual propagations.

‘PAGA 13-1’ exhibits the following distinguishing characteristics when grown under normal horticultural practices in Castroville, Calif.:
1. Large bud size;
2. Primary reproductive shoots produce at least 10 secondary buds, and at least 10 tertiary buds;
3. Plants produce an average of 6 reproductive shoots, in addition to the primary reproductive shoot, during the first production cycle;
4. Bracts and receptacles are wide, and thick;
5. Large diameter peduncle; and
6. Late maturity.

### DESCRIPTION OF THE DRAWINGS

The accompanying photographs illustrate the overall appearance of the new artichoke variety ‘PAGA 13-1’ showing the colors as true as is reasonably possible with colored reproductions of this type. Colors in the photographs may differ slightly from the color values cited in the detailed botanical description, which accurately describes the color of this variety. The depicted plant and plant parts of the new artichoke variety ‘PAGA 13-1’ are from plants that are 5-6 months old, grown in Castroville, Calif.

FIG. 1 shows various sized capitula (buds) of ‘PAGA 13-1’.

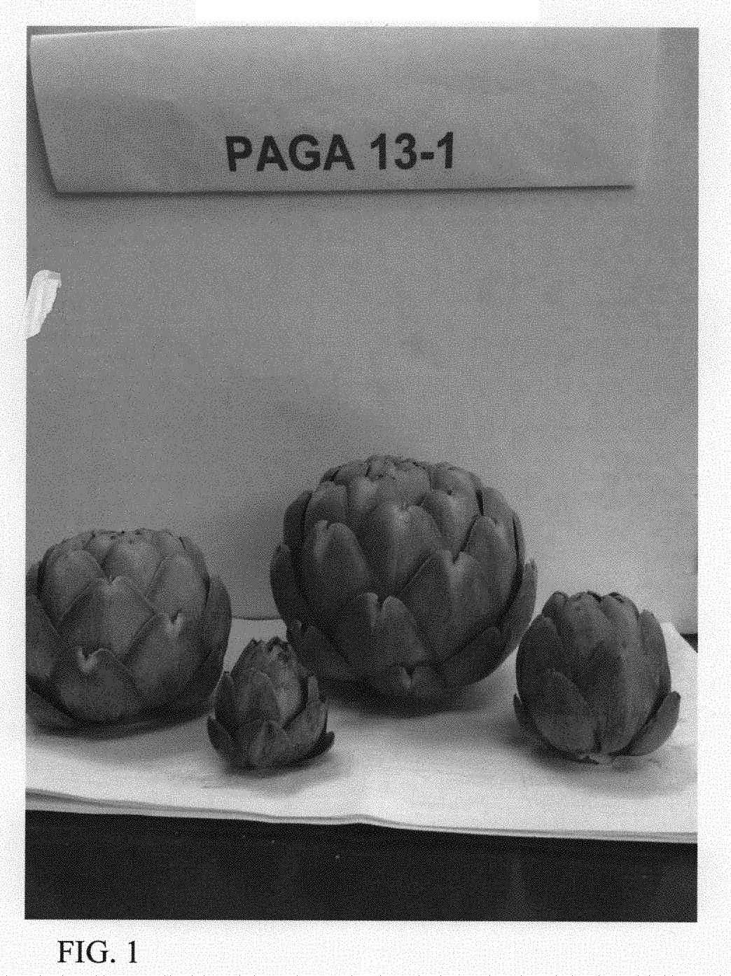

FIG. 2 shows an interior view of a bract of ‘PAGA 13-1’.

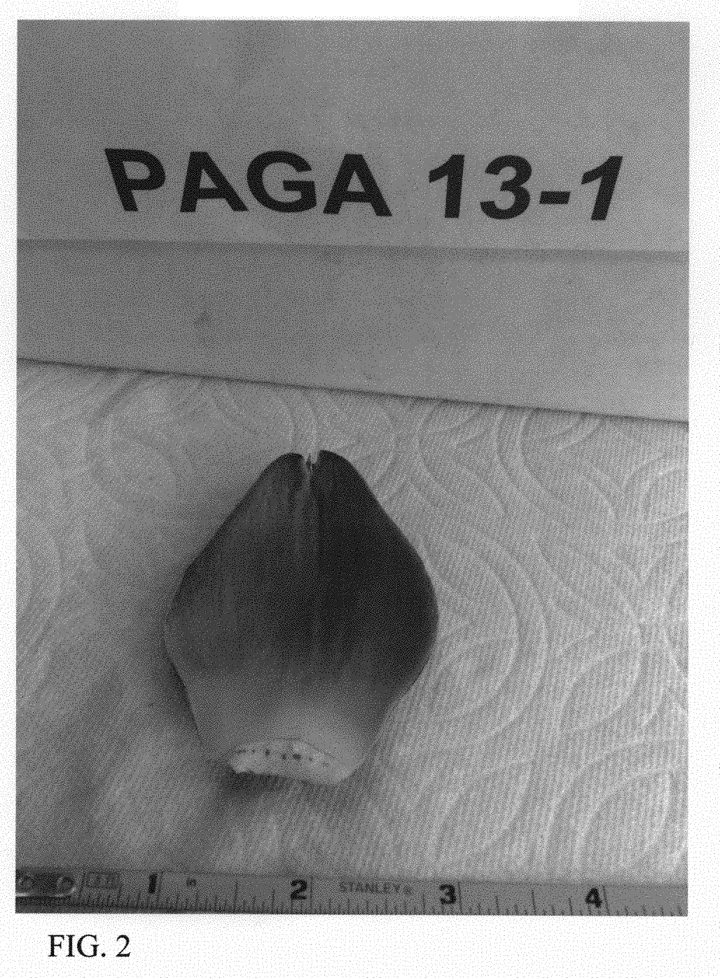

FIG. 3 shows an exterior view of a bract of ‘PAGA 13-1’.

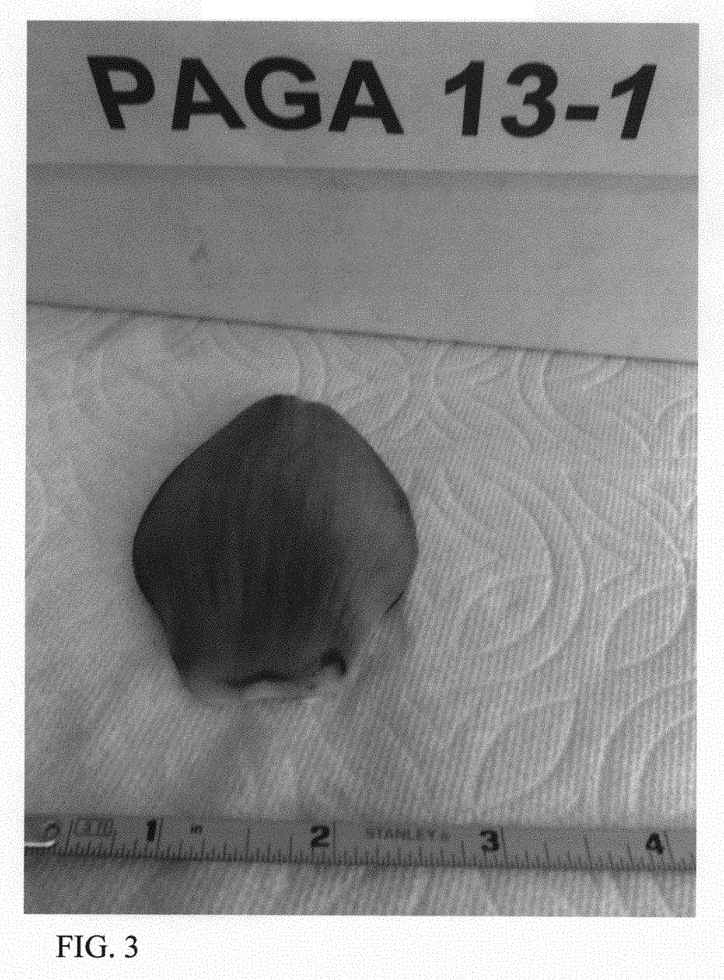

FIG. 4 shows a cross section view of various sized capitula (buds) of ‘PAGA 13-1’.

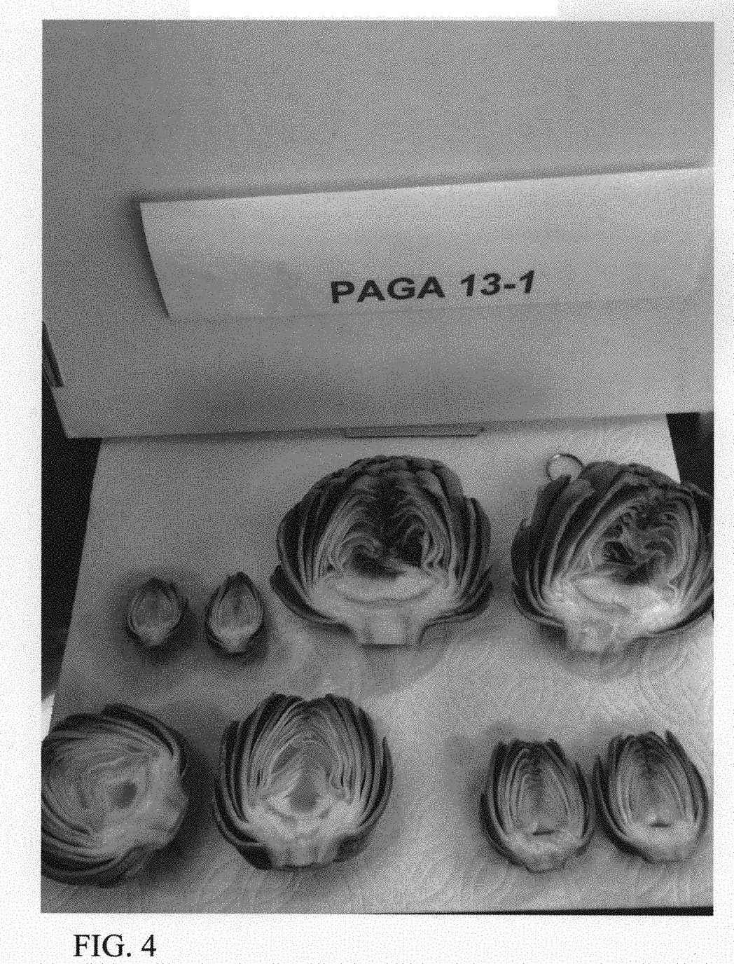

FIG. 5 shows various sized outer and inner bracts of ‘PAGA 13-1’.

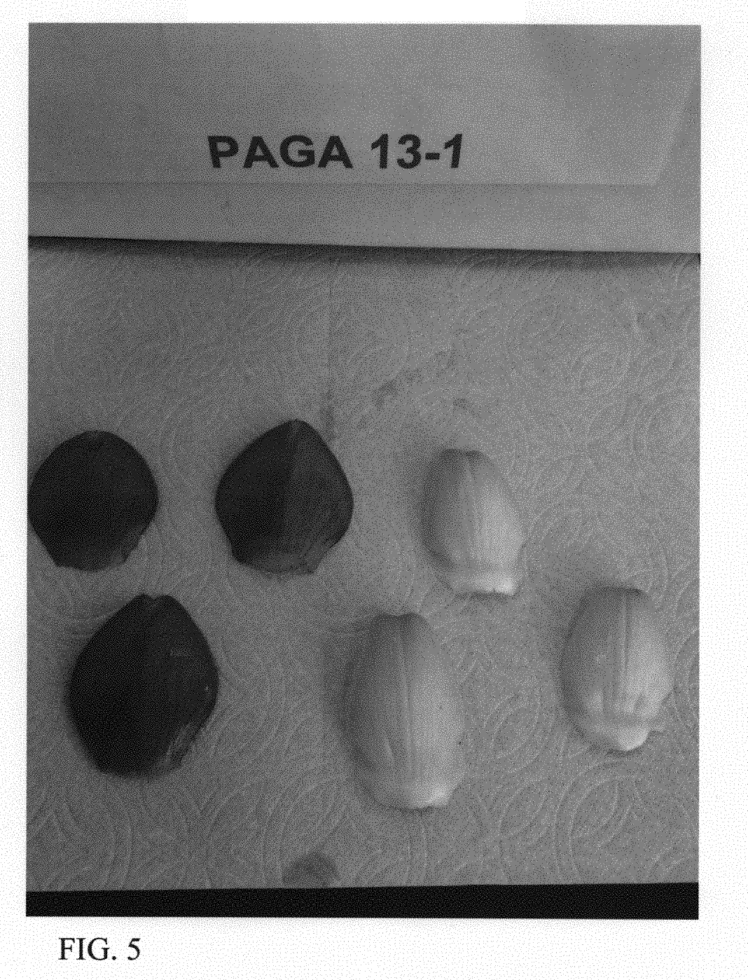

FIG. 6 shows a cauline and a basal rosette leaf of ‘PAGA 13-1’.

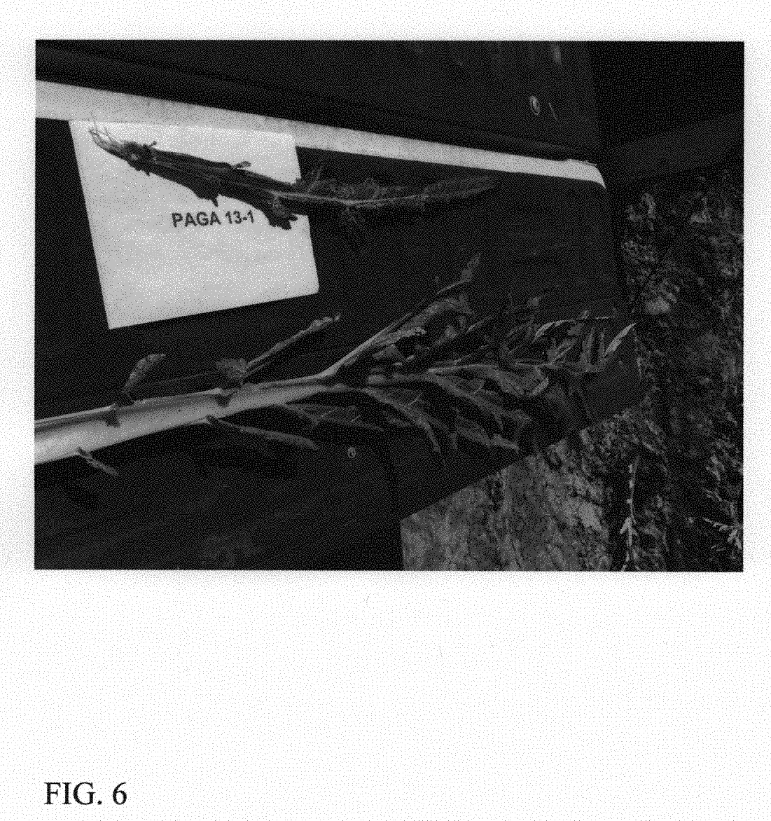

FIG. 7 shows a growing field of ‘PAGA 13-1’ demonstrating its uniformity.

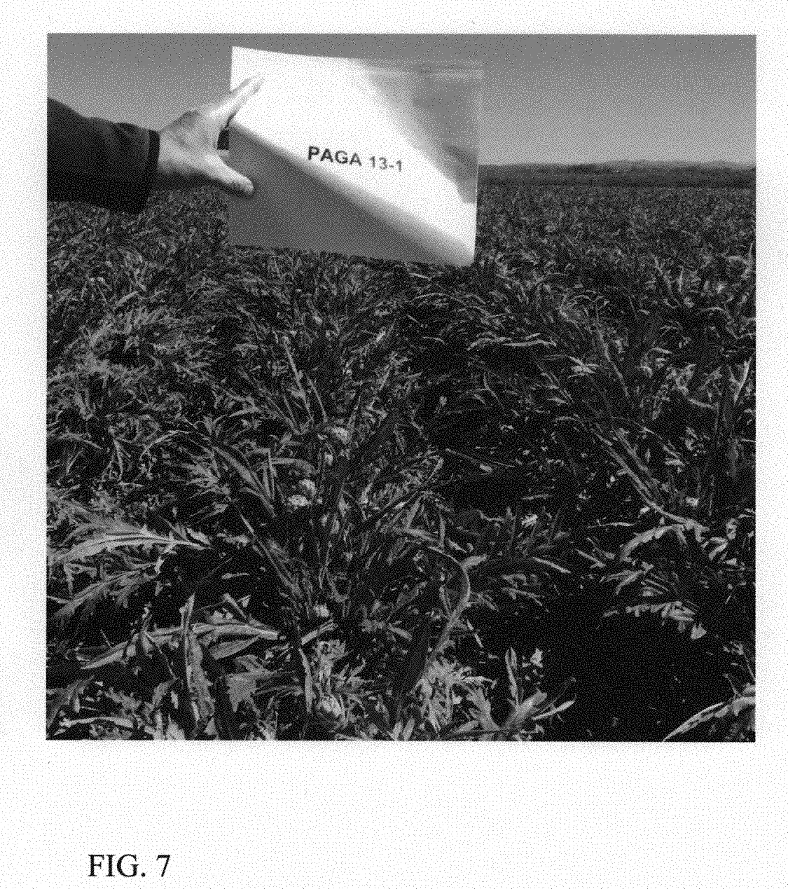

FIG. 8 shows a growing field of parent variety ‘Green Globe’ demonstrating its lack of uniformity.

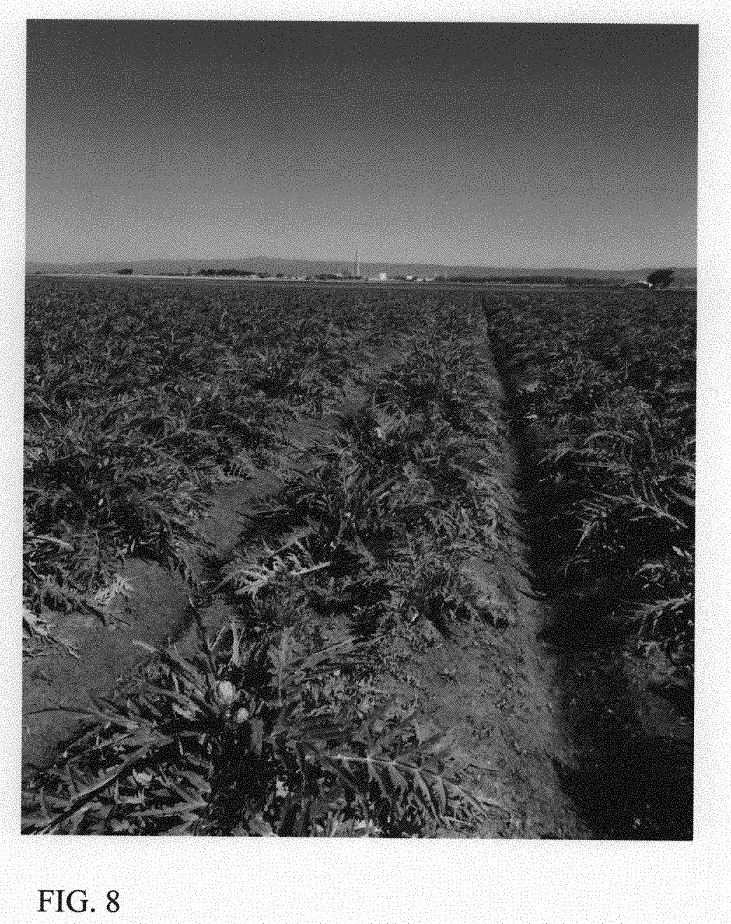

FIG. 9 shows ‘PAGA 13-1’ capitulum (bud) bases showing the differences in purple colored striations on lower bracts.

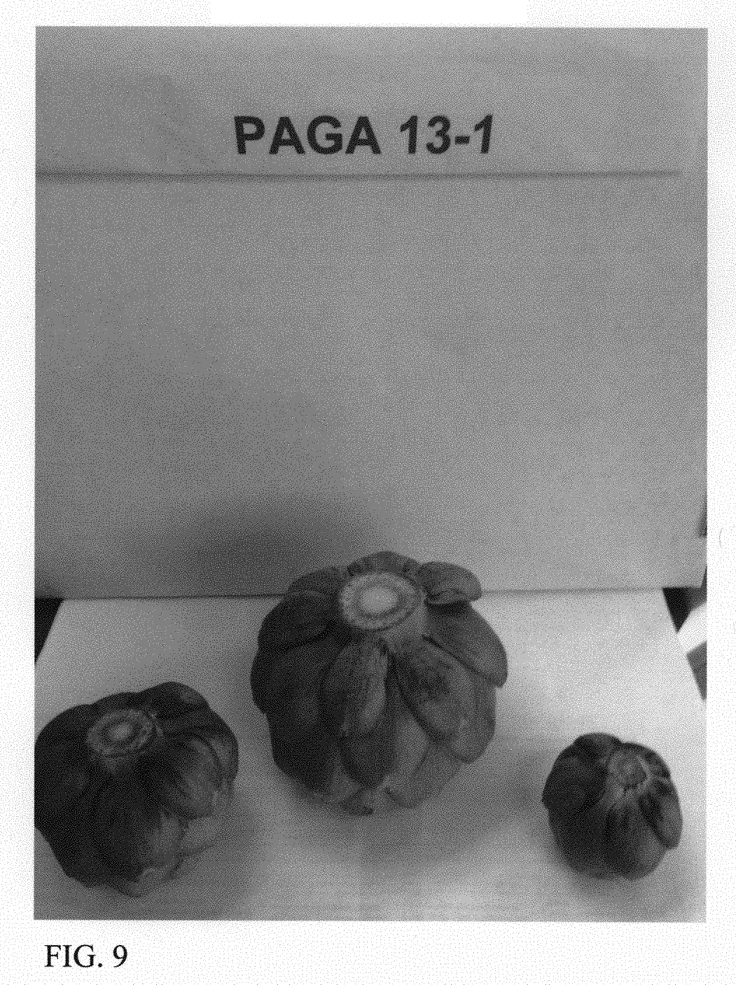

FIG. 10 shows ‘PAGA 13-1’ (right) and ‘Green Globe’ (left) reproductive stems that have been stripped of leaves.

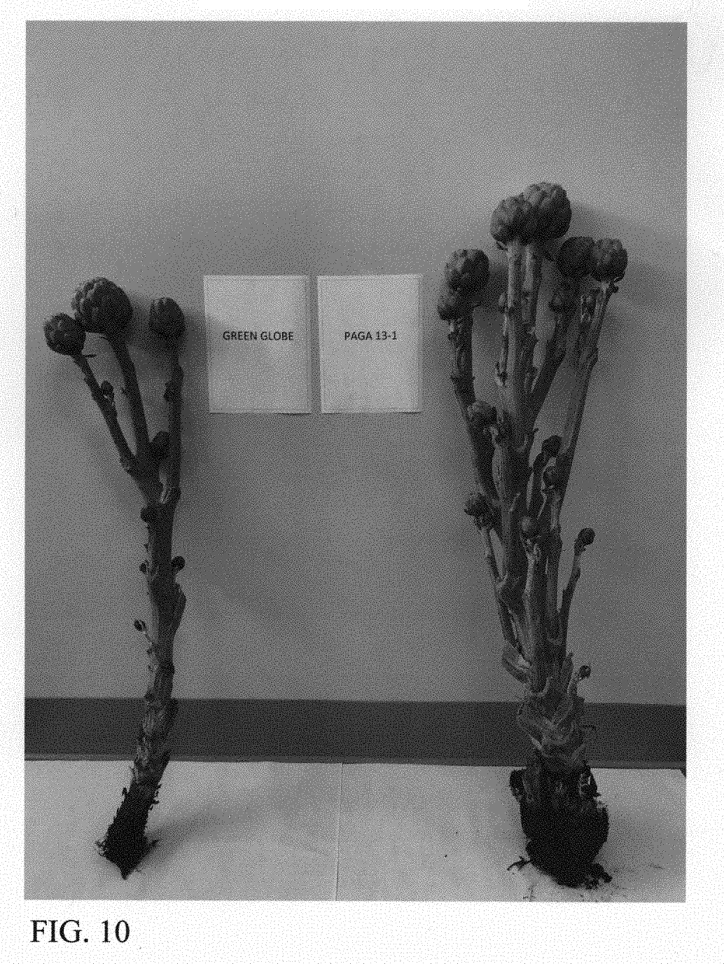

### DETAILED BOTANICAL DESCRIPTION

The following detailed descriptions set forth the distinctive characteristics of ‘PAGA 13-1’. The data which define these characteristics is based on observations taken in Castroville, Calif. This description is in accordance with UPOV terminology. Color designations, color descriptions, and other phenotypical descriptions may deviate from the stated values and descriptions depending upon variation in environmental, seasonal, climatic, and cultural conditions. ‘PAGA 13-1’ has not been observed under all possible environmental conditions. The botanical description of ‘PAGA 13-1’ was taken from plants that were 5-6 months old. The indicated values represent averages calculated from measurements of several plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.
Classification:
   Taxonomic name.—Cynara cardunculus L. var. scolymus.
   Common name.—Globe Artichoke.
   Denomination.—‘PAGA 13-1’.
Parentage: Cynara cardunculus var. scolymus L. ‘PAGA 13-1’ was discovered through successive plant selections, and tissue culture propagation cycles of Cynara cardunculus L. var. scolymus variety ‘Green Globe’.
Plant characteristics table:

| | |
|---|---|
| PAGA 13-1 | Green Globe |

General information

| | | |
|---|---|---|
| Propagation Method: | Tissue culture | Crown division |
| Months of harvest: | Year round with greatest | Year round with greatest |
| | productivity April through July | productivity February through June |
| Number of days from | Range: 165-203 Average: 190 | Range: 161-212 Average 195 |
| planting to first harvest | (tissue culture) | (crown division) |
| Duration of harvest cycle: | Average: 69 days | Average: 232 days |

Plant

| | | |
|---|---|---|
| Height range at harvest | 162.56-198.12 cm (64-78 inches) | 119.38-167.64 cm (47-66 inches) |
| (vegetative): | | |
| Height average at harvest | 185.42 cm (73 inches) | 147.32 cm (58 inches) |
| (vegetative): | | |
| Width at harvest (vegetative): | 152.4-284.48 cm (60-112 inches) | 114.3-132.08 cm (45-52 inches) |
| Growth Habit: | Upright, with horizontal spread of | Upright, with horizontal spread of |
| | older leaves and shoots | older leaves and shoots |
| Form: | Arch | Arch |
| Vigor: | Very Good | Moderate |
| Root system: | Thick and fibrous | Thick and fibrous |
| Foliage Density: | Dense, increasing as secondary | Moderately dense |
| | reproductive shoots develop | |

Primary Reproductive Shoot

| | | |
|---|---|---|
| Height to primary (terminal) | 162.56-198.12 cm (64-78 inches) | 106.68-137.16 cm (42-54 inches) |
| bud top: | | |
| Number of leaves (rosette) | 25-33 (average 31) | 14-28 (average 20) |

Secondary Reproductive Shoots

| | | |
|---|---|---|
| Number per plant in first | 4-10 (average 6) | 1-2 (average 1) |
| production cycle: | | |
| Height to primary (terminal) | 40.64-121.92 cm (16-48 inches) | 13.97-76.2 cm (5.5 to 30 inches) |
| bud top: | | |
| Number of leaves | 5-27 (average 7) | 3-16 (average 7.5) |

Foliage

| | | |
|---|---|---|
| Venation: | Reticulate | Reticulate |
| Leaf ratio (L/W): | Approximately 1.1-3.23 | Approximately 1.12-2.28 |
| Upper leaf surface color: | RHS 143A-143B (Green Group) | RHS 141A-141B (Green Group) |
| Lower leaf surface color: | RHS 141D (Green Group) | RHS 136D (Green Group) |
| Leaf texture: | Glabrous and slightly glossy | Glabrous and slightly glossy |
| | appearance in cool, damp | appearance in cool, damp |
| | conditions with interveinal texture. | conditions with interveinal texture. |
| Pubescence: | Slightly tomentose on upper | Slightly tomentose on upper |
| | surface, moderately tomentose on | surface, moderately tomentose on |
| | lower. More visible in dry/warm | lower. More visible in dry/warm |
| | conditions. | conditions. |
| Petiole color: | RHS 142B-142C (Green Group) | RHS 142C-142D (Green Group) |
| Petiole and mid rib texture: | Furrowed on upper side with lower | Furrowed on upper side with lower |
| | side deeply ribbed with ridges | side deeply ribbed with ridges |
| | running parallel to the lengthwise | running parallel to the lengthwise |
| | orientation. Sparsely arachnoid. | orientation. Sparsely arachnoid. |
| 1. Basal rosette leaves | | |
| Shape: | Deeply pinnatifid with sparsely | Deeply pinnatifid with sparsely |
| | ciliate margins, narrowly trullate | ciliate margins, narrowly trullate |
| | overall. Narrowly acuminate apex | overall. Narrowly acuminate apex. |
| | is less acute than Green Globe. | |
| Size: | Very large, >198.12-78.74 cm | Very large, >172.72 × 45.72 cm |
| | (78 × 31 inches) | (68 × 18 inches) |
| Distance between leaf | 6.35-12.7 cm (2½-5 inches) | 6.35-11.43 cm (2½-4½ inches) |
| division: | | |
| Division indentation length: | 16.51-41.275 cm | 16.51-41.275 cm |
| | (6½-16¼ inches) | (6½-16¼ inches) |
| Petiole length: | 30.48-57.15 cm (12-22½ inches) | 30.48-48.26 cm (12-19 inches) |
| Petiole width: | 3.175-9.525 cm (1¼-3¾ inches) | 3.175-6.985 cm (1¼-2¾ inches) |
| Petiole thickness: | 0.635-1.905 cm (¼-¾ inches) | 0.635-1.905 cm (¼-¾ inches) |
| 2. Cauline leaves | | |
| Shape: | Ensiform overall, uneven and | Ensiform overall, uneven and |
| | sparse serrations with narrowly | sparse serrations with narrowly |
| | acuminate apex. Somewhat | acuminate apex. Somewhat |
| | undulate and dente and sparsely | undulate and dente and sparsely |
| | ciliate at margins. | ciliate at margins. |
| Number of leaves: | Approximately 93 | Approximately 73 |
| Size: | 17.78-60.96 cm (7-24 inches) | 17.78-60.96 cm (7-24 inches) |

Stem (primary reproductive stem system)

| | | |
|---|---|---|
| Length (ground level to base | 162.56-198.12 cm (64-78 inches) | 106.68-127.0 cm (42-50 inches) |
| of terminal capitulum (bud)): | | |
| Circumference: (base of | 7.62-13.97 cm (3-5½ inches) | 7.62-12.065 cm (3-4.75 inches) |
| terminal capitulum (bud): | | |
| Circumference (base of | Average: 36.83 cm | Average: 24.13 cm |
| stem): | (14½ inches) | (9½ inches) |
| Exterior texture: | Vertically ribbed, pubescent | Vertically ribbed, pubescent |
| Stem habit: | Upright, caulescent | Upright, caulescent |
| Pubescence: | Sparsely arachnoid | Sparsely arachnoid |
| Color: | RHS 142A-142B | RHS 142B-142C |

Involucre bracts

| | | |
|---|---|---|
| Arrangement: | Imbricate | Imbricate |
| Quantity per Capitulum | 62 outer-37 inner | 52 outer-35 inner |
| (average): | | |

Outer bract

| | | |
|---|---|---|
| Shape: | Predominately ovate with | Predominately ovate with |
| | flattened base | flattened base |
| Apex: | Retuse with the tips curving down | Retuse |
| | inside the capitulum | |
| Margin: | Entire | Entire |
| Base: | Slightly rounded | Slightly rounded |
| Texture: | Firm, thick and fleshy | Firm, thick and fleshy |
| Color: | RHS 142A-142B (Green Group) | RHS 141C-141D (Green Group) |
| Surface (outer/inner): | Glabrous | Glabrous |
| Spinosity: | Moderate frequency, woody | Moderate frequency, woody |
| Length of spines: | 1-5 mm (0.04-0.2 inches) | 1-5 mm (0.04-0.2 inches) |
| Striation at base of lower | Minor—present on lower outside | Present on approximately 20% of |
| bracts: | bracts | lower outside bracts |
| Striation color: | RHS 77A (Purple-Violet Group) | RHS 77A (Purple-Violet Group) |

Inner bract

| | | |
|---|---|---|
| Shape: | Lanceolate with caudate tip | Lanceolate with caudate tip |
| Apex: | Retuse with the tips curving down | Retuse |
| | inside the capitulum | |
| Margin: | Entire | Entire |
| Texture: | Firm, thinner than outer bract, | Firm, thinner than outer bract, |
| | fleshy | fleshy |
| Base: | Slightly rounded | Slightly rounded |
| Color: | RHS 150B-150C (Green Group) | RHS 150B-150C (Green Group) |
| Surface (outer/inner): | Glabrous | Glabrous |
| Spinosity: | Finer than outer bract, more | Finer than outer bract, more |
| | frequent | frequent |
| Length of spines: | 1-2 mm (0.04-0.079 inches) | 1-2 mm (0.04-0.079 inches) |

Capitulum (head/bud)—when ready for harvest as edible bud

| | | |
|---|---|---|
| Color: | RHS 142A-142B (Green Group) | RHS 141C-141D (Green Group) |
| Shape: | Sub-globose with slightly flattened | Globose to ovoid |
| | base and top | |
| Apex: | Flat to concave | Broadly rounded to obtuse |
| Base: | Truncate to rounded | Truncate to rounded |
| Form: | Tightly compact | Tightly compact |
| Texture: | Firm | Firm |
| Receptacle shape: | Concavo-convex | Concavo-convex |
| 1. Primary (Terminal) | | |
| Capitulum | | |
| Number per primary shoot | 1 | 1 |
| Circumference: | 32.385-38.1 cm (12¾-15 inches) | 32.385-38.1 cm (12¾-15 inches) |
| Weight: | Average 425 grams (0.94 lbs.) | Average 425 grams (0.94 lbs.) |
| Height: | Average 10.16 cm (4 inches) | Average 10.16 cm (4 inches) |
| Diameter: | 11.43-12.7 cm (4½-5 inches) | 11.43-12.7 cm (4½-5 inches) |
| 2. Secondary capitulum | | |
| Number per primary shoot | 10-19 | 3-5 |
| Circumference | 12.065-34.29 cm | 7.62-38.1 cm |
| | (4¾-13½ inches) | (3-15 inches) |
| Weight: | Average 152 grams (0.3351 lbs.) | Average 237 grams (0.52 lbs.) |
| Height: | Average 7.62 cm (3 inches) | Average 8.255 cm (3¼ inches) |
| Diameter: | 2.54-11.43 cm (1-4½ inches) | 2.54-12.7 cm (1-5 inches) |
| 3. Tertiary capitulum | | |
| Number per primary shoot | 10-16 | 2-5 |
| Circumference | 7.62-13.97 cm (3-5½ inches) | 7.62-27.31 cm (3-10¾ inches) |
| Weight: | 36 grams (0.07936 lbs.) | Average 44 grams (0.0970 lbs) |
| Height : | Average 5.08 cm (2 inches) | Average 6.78 cm (2⅔ inches) |
| Diameter: | Average 5.08 cm (2 inches) | 5.08-10.795 cm (2-4¼ inches) |

Receptacle (heart)—when ready for harvest and edible

| | | |
|---|---|---|
| Shape: | Concavo-convex | Concavo-convex |
| Circumference: | 8.89-25.4 cm (3½-10 inches) | 8.89-25.4 cm (3½-10 inches) |
| Color: | RHS 150D (Green Group) | RHS 150D (Green Group) |
| Texture: | Firm | Firm |
| Size: | Very thick and wide when | Very thick and wide when |
| | compared to other globe varieties | compared to other globe varieties |

Peduncle

| | | |
|---|---|---|
| Shape: | Columnar | Columnar |
| Length: | 2.54-101.6 cm (1-40 inches) | 2.54-76.2 cm (1-30 inches) |
| Diameter: | .635-4.445 cm (¼-1¾ inches) | .635-3.81 cm (¼-1½ inches) |
| Color: | RHS 142A-142B (Green Group) | RHS 142B-142C (Green Group) |
| Strength: | Strong and rigid | Strong and rigid |
| Surface: | Glabrous, slightly pubescent with | Glabrous, slightly pubescent with |
| | shallow vertical ribbing | shallow vertical ribbing |

### COMPARISON WITH PARENTAL AND COMMERCIAL VARIETIES

When artichoke variety ‘PAGA 13-1’ is compared to the parental variety ‘Green Globe’, ‘PAGA 13-1’ produces larger sized secondary and tertiary buds and in greater numbers. Additionally, ‘PAGA 13-1’ exhibits less phenotypic variation when grown in the field.

We claim:

1. A new and distinct variety of artichoke plant named ‘PAGA 13-1’ as shown and described herein.
